(19) United States
(12) Patent Application Publication
(10) Pub. No.: US 2010/0062518 A1
(43) Pub. Date: Mar. 11, 2010

(54) Concentrating White Blood Cells for DNA Extraction from a Leukodepleted Blood Sample

(76) Inventor: Sukanta Banerjee, Pennington, NJ (US)

Correspondence Address: ERIC P. MIRABEL, 35 TECHNOLOGY DRIVE, SUITE 100, WARREN, NJ, 07059, US

(21) Appl. No.: 12/552,468

(22) Filed: Sep. 2, 2009

Related U.S. Application Data
(60) Provisional application No. 61/095,358, filed on Sep. 9, 2008.

(51) Int. Cl. C12N 1/08

(52) U.S. Cl. 435/270

(57) ABSTRACT

Reagents and a method for the pre-concentration of WBCs from leukodepleted blood segment samples are described. The reagent comprises: a lytic reagent, for example, saponin in phosphate buffered saline(PBS), wherein the amount of saponin is in the range of from about 1% to about 10% percent (w/w). The method comprises:
contacting a specific volume the leukoreduced blood segment sample with a specific volume of the lytic regent;
mixing the two so as to selectively lyse RBCs in the leukoreduced whole blood segment sample; subjecting the mixture to centrifugation, so as to separate the mixture into a WBC rich phase and a lysed-RBC phase;
discarding the supernatant containing the lysed RBC phase; and resuspending the WBC rich phase in a specific volume of PBS.

### RELATED APPLICATIONS

This application claims priority to U.S. Provisional Application No. 61/095,358, filed Sep. 9, 2008.

### FIELD OF THE INVENTION

The invention relates to concentrating white blood cells (“WBCs”) from a leukoreduced blood sample, for genotyping of DNA from the WBCs.

### BACKGROUND

Whole blood is generally the least expensive and most readily accessible source for genomic DNA. It has the further advantage of providing immediate visual evidence that a sample of adequate size has been obtained. However, isolating DNA from fresh or frozen blood is difficult, since only 0.1% of blood cells are nucleated white blood cells (4−10×10⁷ /ml)—red blood cells have no nucleus or DNA. For example, 1 μl of lysed human blood contains 35-50 ng DNA amid ˜150 ug of protein, lipids and other components.

A variety of techniques have been developed (see FIG. 1) to isolate DNA from blood. For example, in the most rigorous protocols, several milliliters of whole blood are drawn and then centrifuged to separate blood into plasma, a white blood cell (WBC) rich fraction (buffy-coat), and red blood cell (RBC) rich fraction. The WBC's are first isolated and the DNA is released using detergent lysis, followed by protease treatment and DNA purification using phenol-chloroform extraction, followed by ethanol or isopropanol precipitation of the DNA (Sambrook, J. et al. 1989. Molecular cloning, 2^{nd} Ed Cold Spring Harbor Laboratory Press, Cold Spring Harbor, N.Y.). The simplest reported method of DNA extraction involves boiling 1-3 μl of blood in 50 μl of water for cell lysis and directly using a portion of the lysate for further analysis (Skalnik, D. G. and Orkin, S. Biotechniques 8: 34 (1990)). The most popular and versatile among the currently available methods are the solid phase based separation methods. In these methods whole blood is lysed in the presence of an appropriate buffer that allows the released DNA to selectively adsorb on a given solid phase. This is followed by a wash step which selectively washes away the non-specifically adsorbed components, leaving the adsorbed DNA. Finally the adsorbed DNA is eluted using an appropriate elution buffer. Several solid phase DNA extraction methods have been discussed:

U.S. Pat. No. 6,043,354 (to Hilebrand et al.) discusses simultaneous two step extraction of DNA and RNA using solid phase/buffer combinations.

U.S. Pat. No. 5,523,231 (to Reeve et al.) discusses a method of macromolecule (DNA) recovery from solution containing magnetic beads via induced macromolecule precipitation which creates macromolecule-magnetic bead aggregates, which is then separated and the DNA recovered.

U.S. Pat. No. 5,898,071 (to Hawkins et al.) discusses a method for reversibly and non-specifically binding polynucleotides to a functionalized solid using a combination of chaotropic salt and buffer, followed by recovery of the bound DNA.

U.S. Pat. No. 7,173,125 (to Deggeradal et al.) discusses the use of Isolation of nucleic acids from sample using detergents and magnetic beads.

U.S. Pat. No. 5,234,809 (to Boom et al.) discusses a method for isolating nucleic acids from complex nucleic acid containing starting materials (blood, serum etc.) in a one step method using silica particles and chaotropic salt.

U.S. Pat. No. 5,582,988 (to Backus et al.) discusses a method for selective nucleic acid capture and release using weakly basic polymer and different pH.

U.S. Pat. No. 5,945,525 (to Uematsu et al.) discusses a method for nucleic acid separation using silica coated super-paramagnetic particles.

U.S. Pat. No. 6,027,945 (to Smith et al.) discusses a method for nucleic acid separation using silica coated magnetic particles.

No matter the nature of the protocol, the recovery efficiency and the final yield of the DNA is critically dependent on having sufficient numbers of nucleated cells in the initial blood sample. None of the prior art methods address the problem of dealing with leukodepleted blood samples.

Leukodepletion is a process by which leukocytes (WBCs) are removed from donated blood. It is well established that a majority of febrile nonhemolytic adverse transfusion reactions are mediated by donor leukocytes. The use of leulkoreduced products is thus indicated for multi-transfused patients, patients receiving chemotherapy, patients undergoing bone marrow, renal or peripheral blood progenitor cell transplant, and patients with hematologic malignancies. Current standards also require that, as a minimum, blood selected for transfusion to a patient be checked (phenotyped) to be antigen negative to the existing alioantibodies in the patient's serum. Recently DNA analysis has emerged as a powerful, versatile and cost effective method for blood group antigen phenotype determination (Hashmi, G. et al. Transfusion, 45, May 2005; 680-688; Hashmi, G. et al. Transfusion, 47, April 2007, 736-747). DNA analysis using blood relies on the fact that white blood cells (WBCs) are the only cells in blood carrying genomic DNA. When the starting WBC concentration in whole blood is very low (as in the case of leukodepleted samples), it is difficult to carry out DNA based assays using standard DNA extraction protocols. Highly sensitive quantitative PCR techniques have been utilized to characterize the extracted DNA from leukoreduced blood samples (Lee, T.-H. et al. Transfusion, 42 (1) 87-93 (2002)). However, such assay techniques require sophisticated technicians, use dedicated and expensive instrumentation and typically cannot be multiplexed. There is no currently established method or commercially available kit that can utilize leukodepleted blood as a source of genomic DNA for highly multiplexed genotyping assays.

U.S. Pat. No. 6,670,128 B2 (to Smith, et al.) discusses a method for utilizing spent leukodepletion filter devices as a source material for the isolation and analysis of genomic DNA. However, leukodepletion is routinely carried out only in larger blood centers and hence such leukocyte loaded filter devices are only available at a limited number of facilities. In most places, leukodepleted donated blood is available stored in a soft plastic blood collection bag. Because of potential contamination of the blood that may occur from contact with a syringe or pipette used to withdraw a sample, the blood collection bag is connected to a flexible plastic tube that is heat sealed into a series of segments containing the donor's blood. These sealed tube segments are commonly referred to as segment tubes, pigtails, or segments. The segment tubes remain attached to the blood collection bag, and are often folded into a group held together with a rubber band. Whenever the blood is to be tested, the laboratory technician simply removes one or more of the segment tubes attached to the blood collection bag for testing. Since the volume of leukodepleted blood available from the segments is limited such segment samples cannot be utilized for extracting genomic DNA using the filtration device based recovery process as described in U.S. Pat. No. 6,670,128. It has been estimated that the average content of WBCs in donated human whole blood is 10⁹/unit. By the current standards, the total content of WBCs in a leukodepleted blood unit should be less than 5×10⁶/unit or ˜10 WBC's/μl. While it is intuitively clear that theoretically, starting with a 3 log higher volume of blood one can compensate for the leukodepletion, for reasons discussed above, this is not a feasible option. What is required then is a pre-concentration step for the leukocytes from the leukodepleted blood.

Various approaches which allow selective separation and subsequent analysis of WBCs from whole blood are known.

U.S. Pat. No. 5,155,044 (to Ledis et al.) discusses method and reagent system for the rapid isolation, identification and/or analysis of leukocytes from whole blood sample.

U.S. Pat. No. 6,869,798 B2 (to Crews et al) discusses a lytic reagent composition and the method of its use for differential analysis of leukocytes using flow-cytometry.

U.S. Pat. No. 5,789,147 (to Rubenstein et al.) discusses a method for separating high concentrations of WBC's having a high degree of cell viability via low speed centrifugation of blood bags.

U.S. Pat. No. 5,155,044 (to Veriac et al.) discusses a lytic reagent composition for simultaneous measurement of hemoglobin and determination of leukocytes in blood sample comprising a cationic detergent, a compound of the glycoside type and at least one inorganic salt and/or an osmotic and/or leuko-protective agent.

All of the methods described above, though capable of efficient pre-analytic lysing of RBCs for analysis of WBCs using flow cytometry or otherwise, have several deficiencies as far as adaptation of these methods to a WBC pre-concentration method from leukoreduced samples. For example, successful implementation of Veriac et al.'s method (US 5,789,147) requires a high starting blood sample volume ,which is not practical when analyzing leukoreduced segment samples. Use of protocols outlined in U.S. Pat. No. 5,155,044, U.S. Pat. No. 6,869,798 or U.S. Pat. No. 5,155,044 lead to an undesirable high dilution of the recovered intact WBC's, which necessitates further use of lengthy and repeated procedures for re-concentrating the WBC's in a small volume (less than 1 ml) suitable for use in standard solid phase DNA extraction procedures.

Various microfluidic methods have also been described for separation of leukocytes from whole blood (see Sethu et al. Lab Chip, 2006, 6, 83-89, Shevkoplyas et al., Anal. Chem. 2005, 77, 933-937). Such methods, though promising, are not very efficient or easy to use.

WBCs from blood can also be separated using commercially available lymphocyte separation medium (Ficoll-Paque PLUS, GE Healthcare, Piscataway, N.J.). The method involves layering a given volume of whole blood on top of the Ficoll-Paque Plus media and subjecting the mixture to a short, low speed centrifugation. The erythrocytes and the granulocytes sediment to the bottom of the tube and because of their lower density, the lymphocytes, are collected at the interface between the plasma and Ficoll-Paque Plus media. Though this method can be adapted to small volumes of blood, the method is time consuming and rather inefficient in terms of WBC recovery yields (typically less than 30%) when using small volumes (<1 ml).

At present, no method can rapidly and effectively perform the pre-concentration of WBCs from a moderate to small sized leukodepleted blood segment sample with good WBC recovery yield.

### SUMMARY

This invention is directed to reagents and a method for the pre-concentration of WBCs from leukodepleted blood segment samples. The reagents comprise: a lytic reagent containing saponin in phosphate buffered saline(PBS), wherein the amount of saponin is in the range of from about 1% to about 10% percent (w/w).

and the method comprises:

contacting a specific volume the leukoreduced blood segment sample with a specific volume of the lytic regent;

mixing the two so as to selectively lyse RBCs in the leukoreduced whole blood segment sample;

subjecting the mixture to centrifugation, so as to separate the mixture into a WBC rich phase and a lysed-RBC phase;

discarding the supernatant containing the lysed RBC phase; and

resuspending the WBC rich phase in a specific volume of PBS.

This method is a rapid and efficient mechano-chemical method for concentrating and recovering WBCs, with good yields from leukoreduced whole blood segments. The lytic reagent allows one to rapidly and selectively lyse RBCs in a leukoreduced whole blood segment sample while keeping the WBCs intact, without increasing the volume of the leukoreduced sample being lysed significantly.

This method provides an easy, single-step and rapid way to efficiently separate a lysed leukoreduced blood sample into an intact WBC rich phase and a lysed-RBC phase. It further provides that the DNA extracted from the WBC recovered using the reagents and methods outlined herein are of sufficient quality that it does not impair downstream molecular biological analysis, such as DNA polymerase-mediated reactions, RNA polymerase-mediated reactions and Ligase-mediated reactions.

### BRIEF DESCRIPTION OF THE DRAWINGS

FIG. 1 is a summary of genomic DNA extraction techniques.

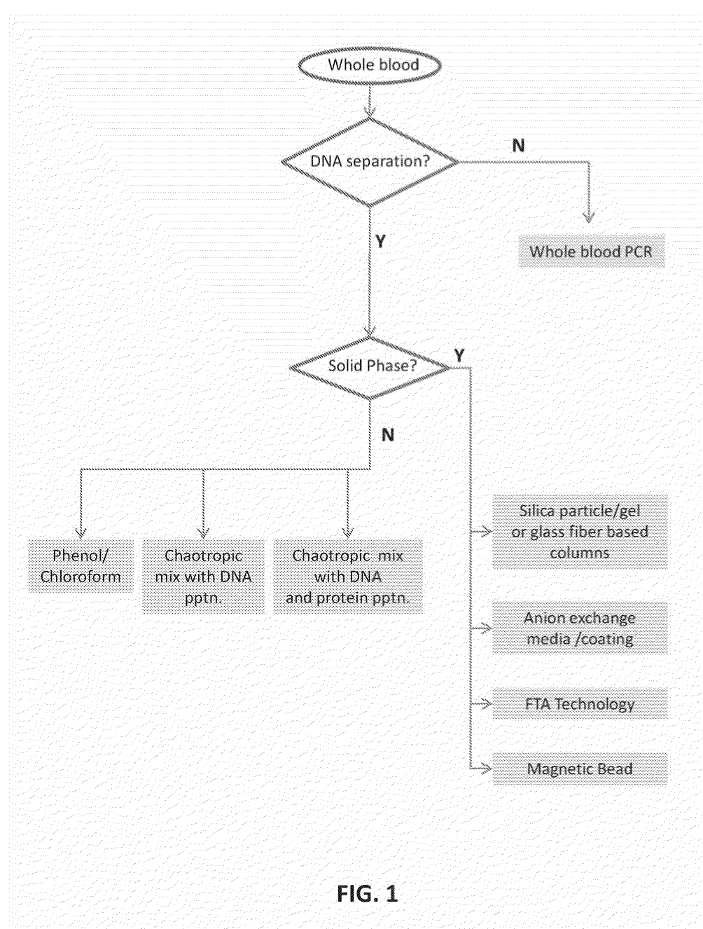

FIG. 2 is a flow chart of the method according to the present invention.

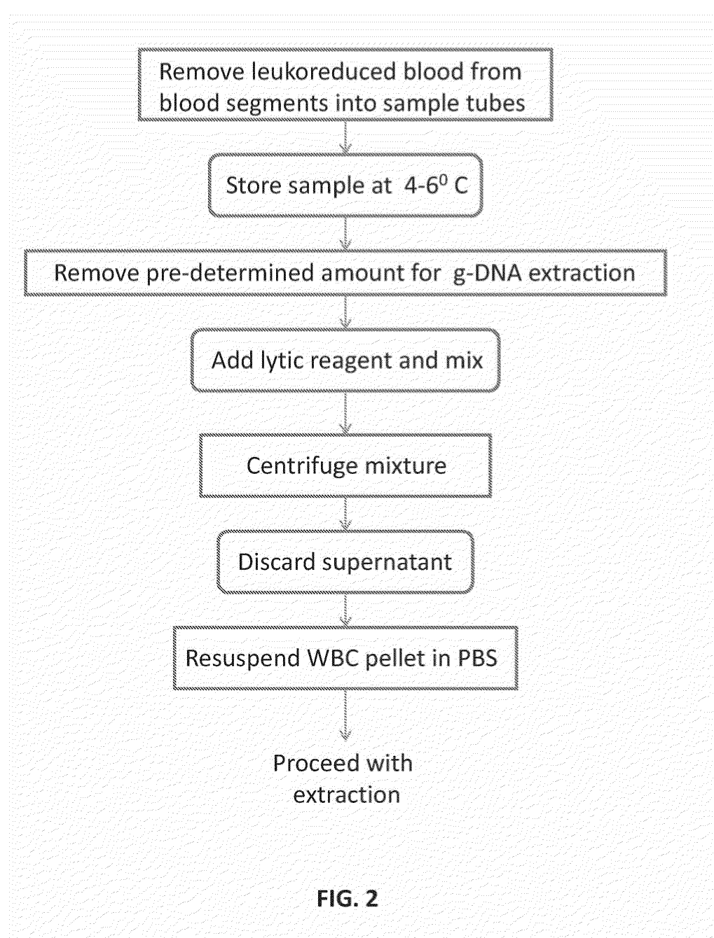

### DETAILED DESCRIPTION

1. The Lytic Reagent

In one embodiment the lytic reagent composition comprises Saponin from Quillaja bark (S 4521, Sigma Aldrich, St. Louis, Mo.) dissolved in PBS buffer. The Saponin is dissolved in PBS at a concentration of about log/L to about 100 g/L. Quillaja saponaria saponin (Quillaja saponins) is a heterogenous mixture of molecules varying both in their aglycone and sugar moieties. The main aglycone (sapogenin) moiety is quillaic acid, a triterpene of predominantly 30-carbon atoms of the Δ12-oleanane type. The aglycone is bound to various sugars including glucose. Quillaja saponin is soluble in water. The solubility in water may be increased by additions of small amounts of alkali. Aqueous solutions of Saponin are known to induce hemolysis of RBCs. At a low concentration, Saponin leads to the formation of a large number of pits in the red blood cell membrane (Seeman, P. et al. J. Cell Biol. 56, 1973, 519-527) whereas at higher concentration, Saponin leads to complete dissolution of the RBCs.

When a blood sample, such as a leukoreduced blood segment sample, is mixed with a sufficient amount of the lytic agent, the RBCs are lysed rapidly while the WBCs remain intact. It has been found that the selection of the buffering agent used for the preparation of the lytic reagent is not critical, as long as the pH is maintained near neutral. It is important to preserve the WBCs under RBC lysis conditions. It has been found that inclusion of physiological levels of saline in the lytic mix prevents undesirable damage to WBCs.

In application, a predetermined volume of blood is mixed with a predetermined volume of lytic agent. The mixing ratio between the blood and lytic agent is generally in the range between 10:1 to about 1000:1.

2. Method for Pre-Concentrating WBCs from a Leukoreduced Whole Blood Segment Sample

In one embodiment, the mechano-chemical method for pre-concentrating WBCs from a leukoreduced whole blood segment sample comprises placing a predetermined volume of leukoreduced blood in a centrifuge tube, adding to it a predetermined volume of lytic agent, mixing the lytic reagent with blood by inverting the tube several times and centrifuging the mix at about 300×g to 1000×g for about 2 min to 6 min, so as to separate the mixture into a WBC rich phase, which pellets at the bottom of the tube, and a lysed-RBC phase, which is present as the supernatant. Following, the supernatant is carefully decanted so as not to disturb the WBC pellet. Subsequently, the WBC pellet is vigorously vortexed and dispersed into a predetermined amount of phosphate buffered saline (PBS), such that the resuspended sample is of adequate volume for use in a solid phase DNA extraction protocol. The volume of PBS employed for re-suspension can be from about 50 μl to about 500 μl. In actuality, the total volume of the resuspended WBC pellet will be somewhat larger than the volume of PBS used for resuspension, because of the residual fluid entrained in the WBC pellet from the centrifugation step. However, small volumes of such carryover fluid have not been found to negatively impact the downstream processes. The resuspended WBCs may be immediately processed for extraction of genomic DNA. Optionally, the resuspended WBCs can be frozen and stored till further use.

3. Solid Phase Method for Genomic DNA Extraction

In one preferred embodiment, 50 μl to 200 μl of recovered WBC suspension is used for genomic DNA extraction using the automated QIAcube instrument (QIAGEN Inc., Valencia, Calif.). The QIAcube is a plug-and-play instrument which simplifies and streamlines the genomic DNA purification procedures by fully automating the spin-column based DNA extraction protocol. The instrument is capable of handling 12 samples per run.

In another preferred embodiment the X-tractor Gene automated nucleic acid extraction instrument (Corbett Life Science, Sydney, Australia) is used for genomic DNA extraction from the recovered WBC suspension. The instrument is capable of handling 96 samples per run.

4. Multiplexed PCR Amplification and Genotyping

In one preferred embodiment the extracted genomic DNA is analyzed for single nucleotide polymorphisms (SNPs) associated with 24 antigens of 10 blood group systems using an HEA BeadChip™ kit (BioArray Solutions Ltd., Warren, N.J.). The data is acquired using an AIS 400 instrument and the analysis carried out using HEA Analysis Software package in the BioArray Solutions Information System (BASIS™) (BioArray Solutions Ltd., Warren, N.J.). This is a qualitative test. However, if the signal intensity for any specific allele is too low (Low Signal, LS), the genotype assignment for that allele cannot be successfully completed. Thus the presence or the number of LS calls can be used to judge the quality of any particular assay run. LS calls can be triggered by not having target DNA present in sufficient quantity. It is worth while to mention that such LS calls can also be a result of polymerase chain reaction (PCR) failure, due to the presence of PCR impurities in the extracted genomic DNA. Many such inhibitory substances are inherent to whole blood sample (Al-Soud, W. A. J. Clin. Microbiol. 38(1) 345-350 (2000), Al-Soud, W. A. J. Clin. Microbiol. 39(2) 485-493 (2001), Bessetti, J. An Introduction to PCR Inhibitors, Promega Profiles in DNA, 10(1) March 2007). One attribute of the method described herein is that it effectively eliminates the introduction of sources of impurities to the genomic DNA extraction step. This is because during the WBC preconcentration from the whole blood the bulk of red cells, plasma and free hemoglobin (which are known PCR inhibitors) are largely discarded.

### EXAMPLES

### Example 1

A lytic reagent composition was prepared as follows

TABLE 1

| | | |
|---|---|---|
| Sodium phosphate | 0.1 | M |
| Sodium chloride | 0.15 | M |
| Saponin | 5 | g |
| Water | Make up to 100 | ml |

pH of the lytic agent is about 7.2

For long term storage 50 mg of sodium azide (per 100 ml) may be added.

Saponin is Saponin from Quillaja bark (S 4521, Sigma Aldrich, St. Louis, Mo.)

### Example 2

A series of normal and diluted whole blood samples (see table below) were treated using the protocol outlined below. 200 ul of recovered WBC suspension was used for genomic DNA extraction using the automated QIAcube instrument (QIAGEN Inc., Valencia, Calif.). The concentration and the quality of the extracted DNA was estimated by measuring OD₂₆₀ and OD₂₆₀/OD₂₈₀ ratio.

Protocol:
1. Take 1 ml of whole/diluted blood in 2 ml centrifuge tube
2. Add 500 μl PBS
3. Add 40 ul of Lytic reagent (5 g Saponin/100 ml of PBS)
4. Mix contents of the tube by turning the tube end-over-end a few times (˜1 min)
5. Spin the tube @2000×g for 3 min
6. Discard supernatant by decanting
7. Resuspend pellet in required volume of PBS (200 μl) using vigorous vortexing
8. Proceed to Qiagen extraction protocol

One 200 μl sample of the whole blood was run without executing the above pre-concentration protocol as the positive control. The efficiency of the lysis/WBC pre-concentration process can be calculated from the data. The duplicate positive control runs provide an estimate of maximum possible DNA recovery using the QIAcube in absence of pre-concentration (˜40 ng/μl), when 1000 ul of blood is subjected to RBC lysis and the resulting WBC pellet is re-suspended to 200 μl. The expected theoretical concentration enhancement should be 5-fold, while experimentally determined enhancement is ˜2.5 fold, indicating an overall WBC recovery efficiency of 50%.

TABLE 2

| | | WBC | Volume | | | DNA | | |
|---|---|---|---|---|---|---|---|---|
| Sample | Amount | resuspension | taken for | Elution | DNA | quality | | |
| ID | of blood | vol. | extraction | vol. | Conc. | 260/280 | Efficiency | Comments |
| 0647L | 200 ul | 200 ul | 200 ul | 50 ul | 10.6 ng/ul | 1.85 | 25% | In all cases the volume |
| | 400 ul | 200 ul | 200 ul | 50 ul | 45.9 ng/ul | 1.87 | 50% | of the input blood was |
| | 600 ul | 200 ul | 200 ul | 50 ul | 77.8 ng/ul | 1.94 | 60% | adjusted to 1 ml using |
| | 1000 ul | 200 ul | 200 ul | 50 ul | 103 ng/ul | 1.93 | 50% | 1x PBS |
| | 200 ul | n.a. | 200 ul | 50 ul | 43 ng/ul | 1.92 | 100% | Positive control run(s) |
| | | | | | | | | without any pre-lysis |

The experimental data indicates that in order to get the largest benefit from the pre-concentration step the maximum possible starting volume of blood should be used in the protocol.

### Example 3

Three 2 ml samples of whole blood were taken. 1.8 ml of the sample was taken through the pre-concentration protocol outlined in Example 2 (for details see table 3). For each sample, a 200 μl aliquot of the whole blood was run as a positive control (without pre-concentration). Note here the maximum expected concentration enhancement is 9 fold (1.8 ml to 0.2 ml).

TABLE 3

| | | | | | | DNA conc. | |
|---|---|---|---|---|---|---|---|
| | | | | | | (ng/μl) | |
| | | | | | DNA conc. | From 200 μl of | |
| | | Volume | Control | Resuspension | (ng/μl) | resuspended | |
| | | of Lytic | Amount of | volume after | (control) | WBC pellet | Efficiency of |
| Sample | Volume | reagent | whole blood | lysis | From 200 μl | (using 1.8 ml | WBC |
| i.d. | taken | added | (μl) | (μl) | whole blood | whole blood) | concentration |
| 0720s | 1.8 ml | 80 ul | 200 | ~200 | 49 | 165 | 37%* |
| 0728L | 1.8 ml | 80 ul | 200 | ~200 | 66 | 336 | 60% |
| 0726c | 1.8 ml | 80 ul | 200 | ~200 | 46 | 289 | 71% |

*Whole blood sample partially clotted

What is claimed is:

1. A method for extracting genomic DNA from leukoreduced blood samples, the method comprising:
contacting a specific volume the leukoreduced blood sample with a specific volume of a lytic reagent capable of lysing red blood cells;
mixing the two so as to selectively lyse red blood cells in the leukoreduced blood sample;
subjecting the lysed mixture to centrifugation, so as to separate the mixture into a white blood cell rich sediment and a supernatant rich in lysed-red blood cells;
discarding the supernatant;
resuspending the white blood cell rich phase in a specific volume of aqueous buffer; and
extracting DNA from the buffered white blood cell suspension.

2. The method of claim 1, wherein the lytic reagent comprises saponin dissolved in phosphate buffered saline.

3. The method of claim 2 wherein the amount of saponin in the lytic reagent is from about 1% to about 10% percent (w/w).

4. The method of claim 2 wherein the lytic reagent further includes alkali and the pH is near neutral.

5. The method of claim 1, wherein the volume of the leukoreduced blood sample is about 0.2 ml to about 2 ml.

6. The method of claim 5, wherein the volume of the lytic reagent is about 40 μl to about 80 μl.

7. The method of claim 1, wherein the buffer is selected from the group consisting of hydrogenated phosphates of sodium and potassium.

8. The method of claim 1, wherein the centrifugation is done at about 300×g to 2000×g and for about 2 to 6 minutes.

9. The method of claim 1 wherein the centrifugation is at 2000×g for 3 minutes.

10. The method of claim 1, wherein the volume of buffer used for WBC resuspension is from about 50 μl to about 500 μl.

11. The method of claim 1 wherein the resuspension of the white blood cell rich phase in phosphate buffered saline is by vortexing.

12. The method of any of claims 1 to 3 further comprising the step of isolating genomic DNA from the white blood cell rich phase.
